(19) United States
(12) Patent Application Publication
(10) Pub. No.: US 2018/0280288 A1
(43) Pub. Date: Oct. 4, 2018

(54) Natural Teeth Whitener Formulation

(71) Applicant: Drew Hamilton, ORLANDO, FL (US)

(72) Inventor: Drew Hamilton, ORLANDO, FL (US)

(21) Appl. No.: 15/944,774

(22) Filed: Apr. 3, 2018

Related U.S. Application Data
(60) Provisional application No. 62/481,355, filed on Apr. 4, 2017.

(51) Int. Cl. A61K 8/9794; A61Q 11/00; A61K 8/19; A61K 8/34; A61K 8/24; A61K 8/9789; A61K 8/73; A61K 8/60; A61K 8/37

(52) U.S. Cl. CPC A61K 8/9794; A61Q 11/00; A61K 8/19; A61K 8/345; A61K 8/24; A61K 2800/5922; A61K 8/73; A61K 8/60; A61K 8/37; A61K 2800/87; A61K 8/9789

(57) ABSTRACT

A composition for whitening teeth including cocos nucifera, turmeric extract. The composition further includes sodium bicarbonate, aqua, glycerin, sodium pyrophosphate, mentha piperita, xantham gum, polysorbate-20, potassium sorbate, and sodium benzoate. The turmeric extract is configured to allow the composition to be white in color. The composition is a natural and safe teeth whitener.

### CROSS REFERENCE TO RELATED APPLICATION

The present application is related to and claims priority to U.S. Provisional Patent Application No. 62/481,355 filed Apr. 4, 2017, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of oral hygiene products and more specifically relates to teeth whiteners.

### 2. Description of Related Art

Brushing teeth is important for proper oral hygiene. Some people may choose to use whitening products, in combination with brushing teeth, in order to keep their teeth looking healthy and whiter. These whitening products may be painful, inconvenient, and include numerous chemicals. This is undesirable. A suitable solution is desired.

U.S. Pat. No. 3,574,824 to James Echeandia, et al. relates to an anhydrous toothpaste formulation. The described anhydrous toothpaste formulation includes an anhydrous toothpaste base prepared using the following ingredients: (1) an oil having a viscosity of 100 to 300 centipoises, such as mineral oil, light liquid petrolatum thickened to the necessary viscosity, or an antioxidant stabilized vegetable oil; (2) a combination of polyethylene glycols having a viscosity of 2200 to 3400 centipoises with molecular weights ranging from 550 to 6000; (3) a non/toxic, non-ionic emulsifier which is a mixture of glycerides of c12 to c17 fat-forming fatty acids; (4) at least one binding agent selected from polyvinylpyrrolidone having an average molecular weight of 40,000 and a colloidal magnesium aluminum silicate; and (5) a compound having a negative heat of hydration which can be a hexahydric alcohol such as mannitol or inositol. To this base, 30% to 70% of an abrasive may be added. The abrasive may be aluminum hydrate and/or calcium sulfate and can contain a small amount of aluminum silicate. Dentifrice additives such as enzymes, bleaching agents which achieve whitening and brightening effects, fluorides, and the like, which are incompatible with standard hydrous toothpaste formulations will remain stable and active when added to this anhydrous toothpaste base. Taste, consistency and foaming properties of the paste are equivalent to hydrous toothpaste formulations.

### BRIEF SUMMARY OF THE INVENTION

In view of the foregoing disadvantages inherent in the known oral hygiene product art, the present disclosure provides a novel teeth whitener formulation. The general purpose of the present disclosure, which will be described subsequently in greater detail, is to provide a white in color, natural, teeth whitening product.

A composition is disclosed herein. The composition for whitening teeth includes cocos nucifera, turmeric extract. The turmeric extract is configured to allow the composition to be white in color. The composition further includes a mixture of sodium bicarbonate, aqua, glycerin, sodium pyrophosphate, mentha piperita, xantham gum, polysorbate-20, potassium sorbate, and sodium benzoate. The cocos nucifera (organic coconut oil) comprises approximately 70% of the composition. The sodium bicarbonate (baking soda) comprises approximately 7.5% of the composition. The aqua comprises deionized water and may be added to create a paste like texture. The mentha piperita (organic peppermint oil) comprises approximately 1.5% of the composition. The turmeric extract comprises approximately 15% of the composition. The combined amounts of sodium benzoate, potassium sorbate, polysorbate-20, xantham gum, sodium pyrophosphate, glycerin, and aqua is approximately 6% of the composition. The composition is a natural and safe teeth whitener and stain remover.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, composition for whitening teeth, constructed and operative according to the teachings of the present disclosure.

FIG. 1 is a view of a composition table for a composition for whitening teeth, according to an embodiment of the disclosure.

FIG. 2 is a view of a pumping mechanism for dispensing the composition, according to an embodiment of the disclosure.

The various embodiments of the present invention will hereinafter be described in conjunction with the appended drawings, wherein like designations denote like elements.

### DETAILED DESCRIPTION

As discussed above, embodiments of the present disclosure relate to oral hygiene products and more particularly to a teeth whitener formulation as used to improve the whitening of teeth and oral hygiene.

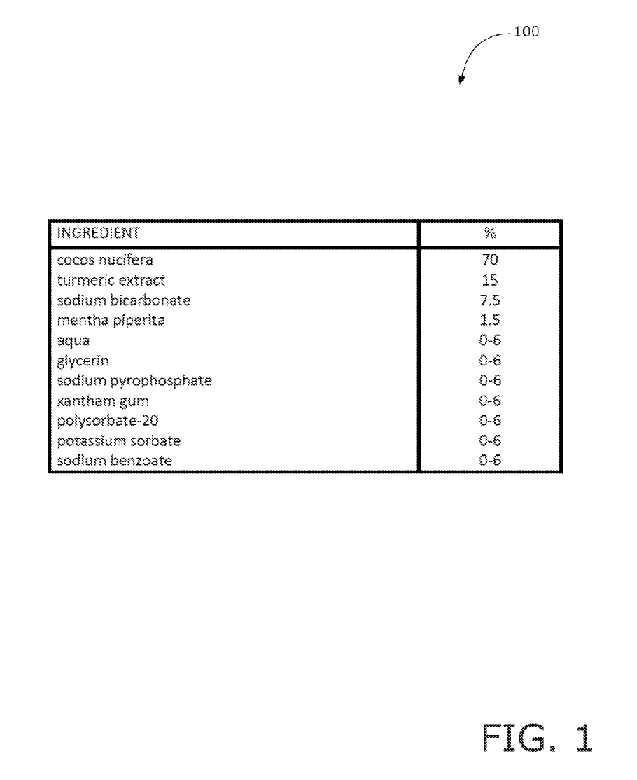

Referring now to FIG. 1, the composition 100 for whitening teeth may include an admixture of cocos nucifera and turmeric extract. The composition 100 further includes sodium bicarbonate, aqua, glycerin, sodium pyrophosphate, mentha piperita, xantham gum, polysorbate-20, potassium sorbate, and sodium benzoate. The cocos nucifera comprises approximately 70% organic coconut oil. The sodium bicarbonate comprises approximately 7.5% baking soda. The aqua comprises deionized water. The mentha piperita comprises approximately 1.5% organic peppermint oil. The turmeric extract comprises approximately 15% of the composition 100. The combined amounts of sodium benzoate, potassium sorbate, polysorbate-20, xantham gum, sodium pyrophosphate, glycerin, and aqua is approximately 6% of the composition 100. Other solutions or products may be used in varying or the same quantities as mentioned herein.

Generally, coconut teeth is an all-natural coconut teeth whitener. The paste is comprised of organic coconut oil (approximately 70%), turmeric extract (approximately 15%), sodium bicarbonate (approximately 7.5%), and peppermint oil (approximately 1.5%) mixed together in a paste form (other ingredients may account for approximately 6% of the total composition). The paste may be placed in various sized containers for dispensing. An air pump style container may be provided for dispensing one pump onto a toothbrush for use. Individuals can use the paste to brush and simultaneously whitening their teeth. The paste is intended to be gentle enough for use on sensitive teeth and gums versus chemical based whitening. The paste may be used to brush the teeth of a user. The paste may be used to whiten teeth using natural and gentle ingredients. Coconut teeth may be edible and be provided in a mini toothpaste tube.

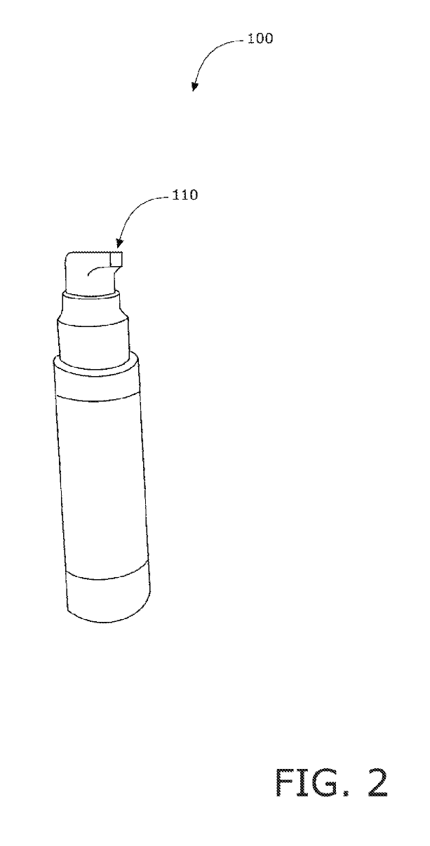

The turmeric extract is configured to allow the composition 100 to be white in color to eliminate stains during use. Prior art mixtures may be colored and easily stain surrounding surfaces such as sinks and countertops during use. The composition 100 is a natural and safe teeth whitener. As shown in FIG. 2, the composition 100 is configured to be dispensable by a pumping mechanism 110. The composition 100 may be dispensed directly onto a tooth brush. A user may simply brush teeth with the formula 2-3 times a week for teeth whitening and dental hygiene improvement results as a method of use. Those with ordinary skill in the art will now appreciate that upon reading this specification and by their understanding the art of toothpaste compositions as described herein, methods of manufacturing and use will be understood by those knowledgeable in such art.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A composition for whitening teeth comprising:
cocos nucifera;
turmeric extract, said turmeric extract configured to allow said composition to be white in color; and
wherein said composition is a natural and safe teeth whitener.

2. The composition of claim 1, wherein said composition further includes sodium bicarbonate.

3. The composition of claim 2, wherein said composition further includes aqua.

4. The composition of claim 3, wherein said composition further includes glycerin.

5. The composition of claim 4, wherein said composition further includes sodium pyrophosphate.

6. The composition of claim 5, wherein said composition further includes mentha piperita.

7. The composition of claim 6, wherein said composition further includes xantham gum.

8. The composition of claim 7, wherein said composition further includes polysorbate-20.

9. The composition of claim 8, wherein said composition further includes potassium sorbate.

10. The composition of claim 9, wherein said composition further includes sodium benzoate.

11. The composition of claim 1, wherein cocos nucifera comprises approximately 70% organic coconut oil.

12. The composition of claim 2, wherein said sodium bicarbonate comprises approximately 7.5% baking soda.

13. The composition of claim 3, wherein said aqua comprises deionized water.

14. The composition of claim 6, wherein said mentha piperita comprises approximately 1.5% organic peppermint oil.

15. The composition of claim 1, wherein said turmeric extract comprises approximately 15% of said composition.

16. The composition of claim 9, wherein the combined amounts of sodium benzoate, potassium sorbate, polysorbate-20, xantham gum, sodium pyrophosphate, glycerin, and aqua is approximately 6% of said composition.

17. The composition of claim 1, wherein said composition is configured to be dispensable by a pumping mechanism.

18. The composition of claim 15, wherein said composition is configured to be dispensed directly onto a tooth brush.

19. A composition for whitening teeth, comprising:
cocos nucifera;
turmeric extract, said turmeric extract configured to allow said composition to be white in color;
wherein said composition is a natural and safe teeth whitener;
wherein said composition further includes sodium bicarbonate;
wherein said composition further includes aqua;
wherein said composition further includes glycerin;
wherein said composition further includes sodium pyrophosphate;
wherein said composition further includes mentha piperita;
wherein said composition further includes xantham gum;
wherein said composition further includes polysorbate-20;
wherein said composition further includes potassium sorbate;
wherein said composition further includes sodium benzoate;
wherein cocos nucifera comprises approximately 70% organic coconut oil;
wherein said sodium bicarbonate comprises approximately 7.5% baking soda;
wherein said aqua comprises deionized water;
wherein said mentha piperita comprises approximately 1.5% organic peppermint oil;
wherein said turmeric extract comprises approximately 15% of said composition;
wherein the combined amounts of sodium benzoate, potassium sorbate, polysorbate-20, xantham gum, sodium pyrophosphate, glycerin, and aqua is approximately 6% of said composition;
wherein said composition is configured to be dispensed by a pumping mechanism; and
wherein said composition is configured to be dispensed directly onto a tooth brush.
